(19) United States
(12) Patent Application Publication
(10) Pub. No.: US 2015/0025437 A1
(43) Pub. Date: Jan. 22, 2015

(54) VASCULAR ACCESS SYSTEM WITH CONNECTOR

(71) Applicant: CryoLife, Inc., Kennesaw, GA (US)

(72) Inventors: Daniel Tomko, Dallas, GA (US); David Gale, Kennesaw, GA (US); Stacy Arnold, Cartersville, GA (US); Venkata Katragadda, Kennesaw, GA (US)

(21) Appl. No.: 14/332,091

(22) Filed: Jul. 15, 2014

Related U.S. Application Data
(60) Provisional application No. 61/847,770, filed on Jul. 18, 2013.

(51) Int. Cl. A61M 1/36; A61M 1/10

(52) U.S. Cl. CPC A61M 1/3655; A61M 1/1008
USPC 604/8

(57) ABSTRACT

A connector for fluidly coupling proximal and distal tubular segments of a fluid conduit is provided. In one embodiment, a system is provided for providing continuous flow of blood between two locations in a patient's cardiovascular system. A first blood conduit is provided that has a distal portion and a proximal portion. The distal portion is adapted to be inserted into a blood vessel at an insertion site and to be advanced therethrough to a location spaced apart from the insertion site. The system is provided with a connector that has a distal portion adapted to be engaged with the proximal portion of the first blood conduit and a tubular body extending proximally of the distal portion. The tubular body is adapted to be inserted into and/or attached to an end of a second blood conduit, such as a vascular graft or the like.

### CROSS-REFERENCE TO RELATED APPLICATIONS

The present disclosure claims priority to and the benefit of U.S. Provisional Application No. 61/847,770, filed Jul. 18, 2013, which is hereby incorporated by reference in its entirety.

### FIELD

The present disclosure generally relates to a connector which can be included in a system for connecting multiple portions of a fluid carrying conduit, such as an implantable vascular conduit.

### BACKGROUND

In the United States, approximately 400,000 people have end-stage renal disease requiring chronic hemodialysis. Permanent vascular access sites for performing hemodialysis may be formed by creating an arteriovenous (AV) anastomosis whereby a vein is attached to an artery to form a high-flow shunt or fistula. A vein may be directly attached to an artery, but it may take 6 to 8 weeks before the venous section of the fistula has sufficiently matured to provide adequate blood flow for use with hemodialysis. Moreover, a direct anastomosis may not be feasible in all patients due to anatomical considerations.

Other patients may require the use of artificial graft material to provide an access site between the arterial and venous vascular systems. Patency rates of grafts are still not satisfactory, as the overall graft failure rate remains high. Temporary catheter access is also an option. However, the use of temporary catheter access exposes the patient to additional risk of bleeding and infection, as well as discomfort.

Vascular access systems are known in the art. For example, U.S. Pat. No. 6,102,884 to Squitieri, U.S. Pat. No. 7,762,977 to Porter, and U.S. Pat. No. 8,079,973 to describe implantable blood conduit systems which include (i) an extravascular blood conduit that has a proximal end adapted to couple with a first vascular segment of a patient and a distal end adapted to be inserted into a second vascular segment of the patient; (ii) a catheter having a proximal portion and a distal portion that, when implanted, floats freely within the second vascular segment; and (iii) a connector for fluidly coupling the proximal end of the blood conduit with the catheter with the proximal portion. It would be desirable to provide improved and/or alternative connectors that can readily be used with a variety of conduits or catheters, provide secure and reliable connections, and that physicians find relatively simple to use during an implantation procedure.

### SUMMARY

Some or all of the above needs and/or problems may be addressed by certain embodiments disclosed herein. For example, in one embodiment, a system is provided for providing continuous flow of blood between two locations in a patient's cardiovascular system. A first blood conduit is provided that has a distal portion and a proximal portion. The distal portion is adapted to be inserted into a blood vessel at an insertion site and to be advanced therethrough to a location spaced apart from the insertion site. The system is provided with a connector that has a distal portion adapted to be engaged with the proximal portion of the first blood conduit and a tubular body extending proximally of the distal portion. The tubular body is adapted to be inserted into and/or attached to an end of a second blood conduit, such as a vascular graft or the like.

Other embodiments, aspects, and features of the disclosure will become apparent to those skilled in the art from the following detailed description, the accompanying drawings, and the appended claims.

### BRIEF DESCRIPTION OF THE DRAWINGS

The structure and method of using the devices, systems, and methods described herein will be better understood with the following detailed description of embodiments, along with the accompanying illustrations, which are not necessarily drawn to scale.

FIG. 1 is a perspective view of an embodiment of a vascular access system.

FIG. 2 is a side view of an embodiment of a connector of a vascular access system.

FIG. 3 is a side view of an embodiment of a connector of a vascular access system.

FIG. 4 is a side view of an embodiment of a connector of a vascular access system.

FIG. 5 is a side view of an embodiment of a connector of a vascular access system.

FIG. 6 is a side view of an embodiment of a connector of a vascular access system.

FIG. 7 is a side view of an embodiment of a connector of a vascular access system.

FIG. 8 is a perspective view of an embodiment of a connector of a vascular access system.

### DETAILED DESCRIPTION

Improved hemodialysis and vascular access systems and methods have been developed. In one broad sense, certain embodiments include a blood flow component that can be used for outflow of blood from the system and a coupling or connecting device that can be used to secure an inflow conduit of the system relative to the outflow conduit. Secure connection of the inflow and outflow components enable a continuous flow through the system, where the inflow and outflow components are in secure fluid communication with each other. Techniques for connecting the outflow component with the connecting device are also provided. In various embodiments, the connective device and assembly methods improve over, and/or build upon, the connecting means described in U.S. Pat. No. 8,079,973 and U.S. Application Publication No. 2013/0060268, which are hereby incorporated by reference in their entirety.

A proximal portion of the connecting device is provided in various embodiments to enable fluid connection of the outflow component with any of a variety of inflow components. The inflow components can be any of a variety of blood conduits that are able to be connected to the vascular system to receive blood into the vascular access system. Such blood conduits can have a construction similar to a vascular graft made of ePTFE, Dacron, or other suitable materials. Suitable materials generally are biocompatible with vascular tissues and have a non- or minimally thrombogenic characteristic. The inflow component preferably is adapted for long term attachment to an artery. The inflow component preferably includes a region suitable for repeated needle access. For example, a length of the inflow component can be configured to be pierced by a needle to enable blood to be withdrawn from and returned to the system. While packaging an outflow component with a connector is convenient to the manufacturer, different end users may have different preferences regarding which inflow component to use. For example, some end users may prefer one material or manufacturer over others for any of a variety of reasons.

Accordingly, to enable the outflow component to be used with a wide variety of grafts and other inflow components, connecting devices have been developed and are provided herein that are connectable to a variety of grafts and other inflow components. Moreover, connecting devices have been developed and are provided herein that are connectable to a variety of catheters and other outflow components.

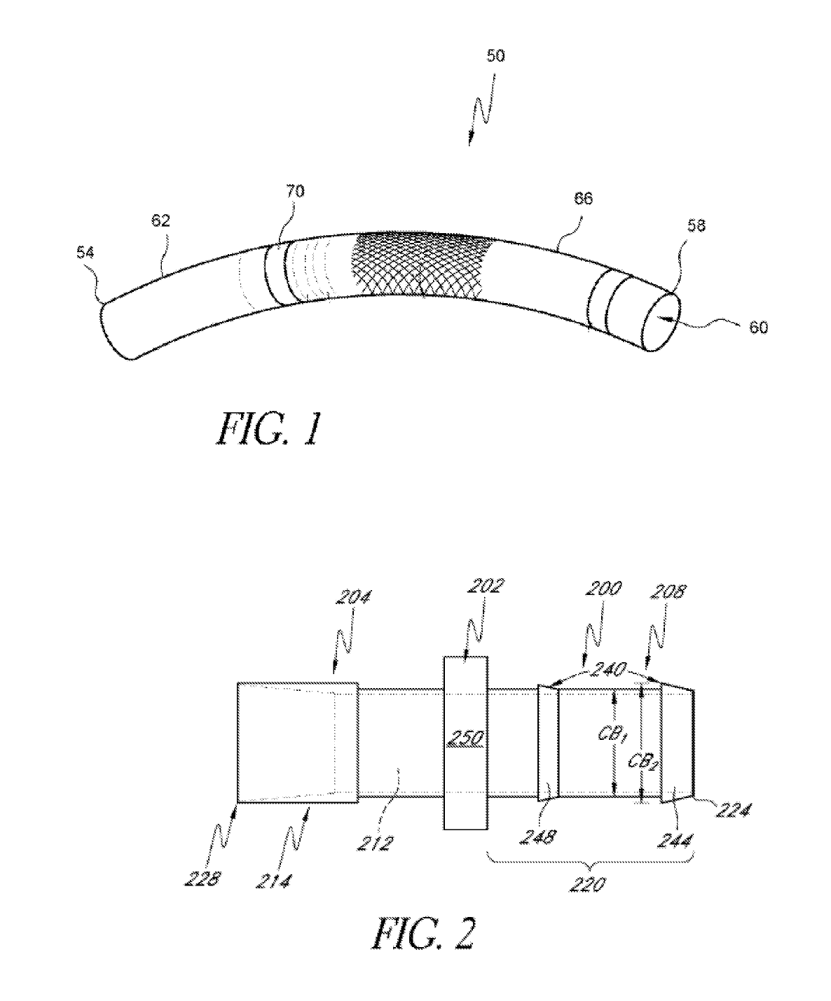

FIG. 1 illustrates one embodiment of a vascular access system 50 having a plurality of components that can be assembled together to form a lumen 60. The lumen 60 provides a blood conduit or pathway configured to shunt blood from a first vascular segment to a second vascular segment. The vascular access system 50 has a proximal end 54 and a distal end 58 and a lumen 60 that extends between the proximal and distal ends 54, 58. In some embodiments, the proximal end 54 can be adapted to couple with (e.g., attached to) a first vascular segment and the distal end 58 can be adapted to be coupled with (e.g., inserted into) a second vascular segment. The lumen 60 preferably extends between the proximal and distal ends 54, 58. The lumen 60 can also be accessed from outside the patient to facilitate dialysis or another medical treatment.

In one embodiment, as illustrated in FIG. 1, the vascular access system includes a connector 70 adapted to fluidly connect a first conduit 62, such as an inflow component or graft, and a second conduit 66, such as an outflow component or catheter, to form the lumen 60. In certain embodiments, the first conduit 62 extends from the proximal end 54 toward the distal end 58, and the second conduit extends from the distal end 58 toward the proximal end 54. The connector 70 can be positioned between the first and second conduits 62, 66 such that a distal portion of the first conduit 62 is configured to be connected to a proximal portion of connector 70 and a proximal portion of the second conduit 66 is configured to be connected to a distal portion of connector 70. The connector 70 and/or conduits 62, 66 can be provided and/or integrated with one or more connecting devices to connect or enhance the security of connection between the first and second conduits 62, 66.

FIG. 2 illustrates one embodiment of a connector 200 that can be incorporated into the blood conduit of the vascular access system 50 of FIG. 1. In certain embodiments, the connector 200 includes a connector body 202 that has a proximal portion 204, a distal portion 208, and a lumen 212 extending therebetween. The lumen 212 can take any suitable form. In one embodiment, the lumen 212 includes a tapered section similar to that described in U.S. Pat. No. 7,762,977, which is hereby incorporated by reference.

The proximal portion 204 preferably is configured to interface with (e.g., be coupled to) the first conduit 62. The connection between the connector 200 and the first conduit 62 can be achieved in any suitable manner. For example, the proximal portion 204 can have an enlarged portion 214, such as a tubular body or the like, over which the conduit 62 can be advanced. In other embodiments, the enlarged portion 214 may be omitted. Other techniques and structures for connecting the connector 200 and the first conduit 62 are described in U.S. Pat. No. 7,762,977; U.S. Application Publication No. 2007/0167901; and U.S. Application Publication No. 2013/0060268, which are hereby incorporated by reference.

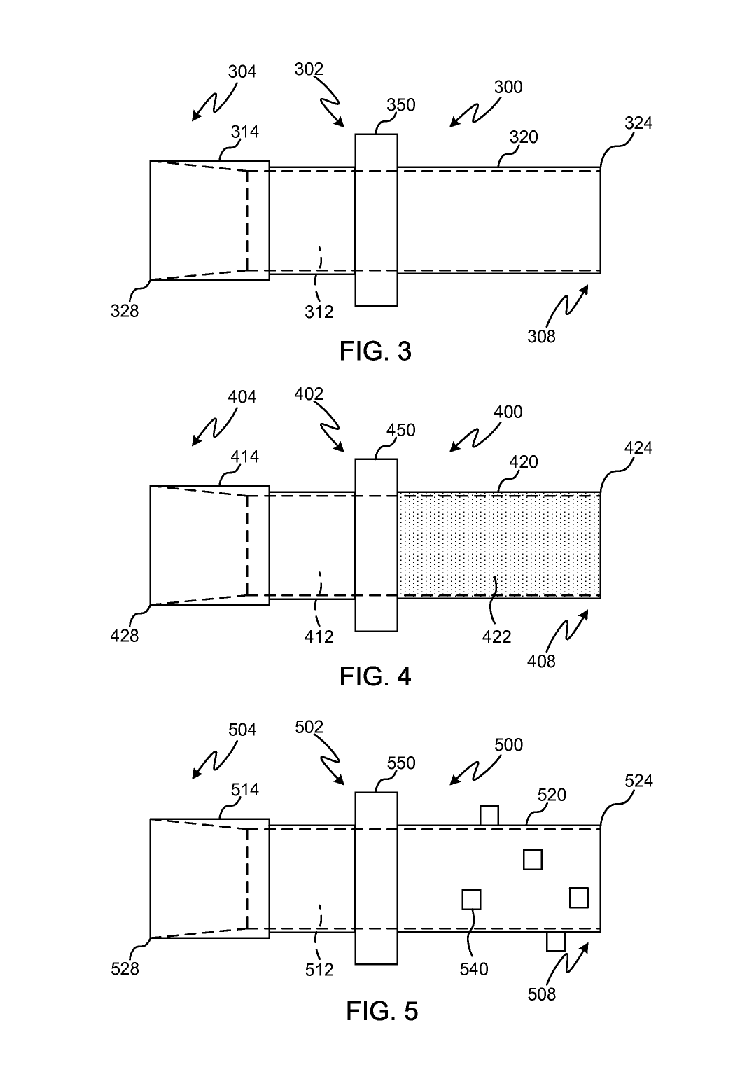

The distal portion 208 is configured to interface with the second conduit 66. In one embodiment, the distal portion 208 includes an outer surface 220 that extends between a distal end 224 and a proximal end 228 of the connector 200. In some instances, the outer surface 220 extends from the distal end 224 to a proximal end of the distal portion 208, adjacent to an enlarged segment 250. The connector 200 also may include an engagement feature 240 that is disposed on the outer surface 220. In one embodiment, the engagement feature 240 includes a portion of an engagement mechanism. As described further herein, the engagement feature 240 may include any number of engagement mechanisms, such as barbs, protrusions, voids, channels, textured surfaces, clamps (integral or extrinsic), sutures, etc. Further, in some instances, the engagement features 240 may be omitted, as depicted in FIG. 3 and elsewhere.

The engagement feature 240 can take any suitable form. For example, in one embodiment, the connector body 202 may include a first outer size CB1, and the engagement feature 240 may include a second outer size CB2 that is greater than the first outer size CB1. In some instances, the outer sizes CB1 and CB2 correspond to diameters. In other instances, the outer sizes CB1 and CB2 correspond to outer perimeters. The diameter of CB1 and CB2 may be any size suitable for performing their intended function.

In certain embodiments, the engagement feature 240 includes one or more barbs disposed about the connector 200 to facilitate a secure connection to, for example, the second conduit 66. In some embodiments, the engagement feature 240 includes a barb 244 that extends over a portion of the connector body 202. The barb 244 can include any structure that includes a raised surface that extends above the connector body. In some instances, a second barb 248 is provided between the first barb 244 and the proximal portion 204 of the connector 200. The second barb 248 of the engagement feature 240 may enhance the security of the connection between the second conduit 66 (e.g., a catheter) and the connector 200. The second barb 248 can take any suitable form. In some embodiments, the second barb 248 is smaller than the first barb 244. In other embodiments, the second barb 248 is larger than the first barb 244. In yet other embodiments, the second barb 248 and the first barb 244 are equal. In still other embodiments, the second barb 248 and/or the first barb 244 are omitted. The height, width, and length of the engagement feature 240 (or barbs) may be any dimension suitable for its intended purpose. In addition, the spacing between the barbs 244, 248 may be any suitable distance for its intended purpose. Further, the distance that the catheter is advanced past the proximal most barb, if present, may be any suitable distance for its intended purpose.

FIG. 3 illustrates one embodiment of a connector 300 that can be incorporated into the blood conduit of the vascular access system 50 of FIG. 1. The connector 300 includes a connector body 302 that has a proximal portion 304, a distal portion 308, and a lumen 312 extending therebetween. The lumen 312 can take any suitable form.

The proximal portion 304 preferably is configured to interface with (e.g., be coupled to) the first conduit 62. The connection between the connector 300 and the conduit 62 can be achieved in any suitable manner. For example, the proximal portion 304 can have an enlarged portion 314 over which the conduit 62 can be advanced. In other embodiments, the enlarged portion 314 is omitted. Other techniques and structures for connecting the connector 300 and the first conduit 62 may be used herein.

The distal portion 308 is configured to interface with the second conduit 66. In one embodiment, the distal portion 308 includes an outer surface 320 that extends between a distal end 324 and a proximal end 328 of the connector 300. In some instances, the outer surface 320 extends from the distal end 324 to a proximal end of the distal portion 308, adjacent to an enlarged segment 350. The connector 300 is similar in many aspects to the connector 200. However, the connector 300 does not include engagement features. That is, the outer surface 320 is uniform and comprises a constant diameter or perimeter. In this manner, a proximal portion of the second conduit 66 may be configured to be slid over the outer surface 320. One or more features, e.g., prestressing or braided tubes, may be associated with the second conduit 66 so as to maintain a secure connection between the connector 300 and the second conduit 66. For example, the second conduit 66 may include one or more attachment features as described in U.S. Pat. No. 8,079,973 and U.S. Application Publication No. 2013/0060268. Other attachments means may be used to secure the second conduit 66 to the connector 300, such as, but not limited to, clamps or the like. In some instances, the distal end 324 may include a taper or the like. For example, the distal end 324 may include a bevel, a chamfer, an angled surface, or the like.

FIG. 4 illustrates one embodiment of a connector 400 that can be incorporated into the blood conduit of the vascular access system 50 of FIG. 1. The connector 400 includes a connector body 402 that has a proximal portion 404, a distal portion 408, and a lumen 412 extending therebetween. The lumen 412 can take any suitable form.

The proximal portion 404 preferably is configured to interface with (e.g., be coupled to) the first conduit 62. The connection between the connector 400 and the conduit 62 can be achieved in any suitable manner. For example, the proximal portion 404 can have an enlarged portion 414 over which the conduit 62 can be advanced. In other embodiments, the enlarged portion 414 may be omitted. Other techniques and structures for connecting the connector 400 and the first conduit 62 may be used herein.

The distal portion 408 is configured to interface with the second conduit 66. In one embodiment, the distal portion 408 includes an outer surface 420 that extends between a distal end 424 and a proximal end 428 of the connector 400. In some instances, the outer surface 420 extends from the distal end 424 to a proximal end of the distal portion 408, adjacent to an enlarged segment 450. The connector 400 is similar to the connector 300. However, the outer surface 420 is textured 422. In this manner, a proximal portion of the second conduit 66 may be configured to be slid over the textured 422 outer surface 420. The textured 422 outer surface 420 may be any textured surface suitable for maintaining an attachment between the proximal portion of the second conduit 66 and the connector 400. All or part of the outer surface 420 may be textured. Moreover, the outer surface 420 may include any textured 422 design/pattern thereon. The textured surface 422 may be created, for example, by molding, machining, and/or fabricating a pattern of grooves, cross-hatches, or random roughened features into and/or onto the surface. Any manufacturing technique or the like may be used to create the textured surface 422. Other attachments means described herein may be used in combination with the textured 422 outer surface 420 to secure the second conduit 66 to the connector 400. In some instances, an inner surface of the proximal portion of the second conduit 66 includes a corresponding textured surface to facilitate attachment to the connector 400.

The term “textured” in this application is not necessarily specific to a predetermined surface roughness, although it could be. Rather, in some instances, it is intended to mean that a condition could be imparted to a surface to provide adequate friction to resist separation of the conduit from the connector. The scale of this texture could range from nano texture, such as may be the result of a chemical etching process, to micro texture, such as residual machine marks, to a macro texture, such as may be achieved by knurling or teeth (as on a file or rasp).

FIG. 5 illustrates one embodiment of a connector 500 that can be incorporated into the blood conduit of the vascular access system 50 of FIG. 1. The connector 500 includes a connector body 502 that has a proximal portion 504, a distal portion 508, and a lumen 512 extending therebetween. The lumen 512 can take any suitable form.

The proximal portion 504 preferably is configured to interface with (e.g., be coupled to) the first conduit 62. The connection between the connector 500 and the conduit 62 can be achieved in any suitable manner. For example, the proximal portion 504 can have an enlarged portion 514 over which the first conduit 62 can be advanced. In other embodiments, the enlarged portion 514 is omitted. Other techniques and structures for connecting the connector 500 and the conduit 62 may be used herein.

The distal portion 508 is configured to interface with the second conduit 66. In one embodiment, the distal portion 508 includes an outer surface 520 that extends between a distal end 524 and a proximal end 528 of the connector 500. In some instances, the outer surface 520 extends from the distal end 524 to a proximal end of the distal portion 508, adjacent to an enlarged segment 550. The connector 500 includes one or more protrusions 540 extending from the outer surface 520. In some instances, a plurality of protrusion 540 extends from the outer surface 520. Each protrusion 540 of the plurality of protrusions 540 may be discrete from one another. In one embodiment, the protrusions 540 are aligned in a uniform manner about the outer surface 520. In another embodiment, the protrusions 540 are disposed about the outer surface 520 in a non-uniform (intermittent) manner. The size, position, and shape of the protrusions 540 may vary. In this manner, a proximal portion of the second conduit 66 may be configured to be slid over the protrusions 540 about the outer surface 520. The protrusions 540 may facilitate maintaining an attachment between the proximal portion of the second conduit 66 and the connector 500. Other attachments means described herein may be used in combination with the protrusions 540 to secure the second conduit 66 to the connector 500.

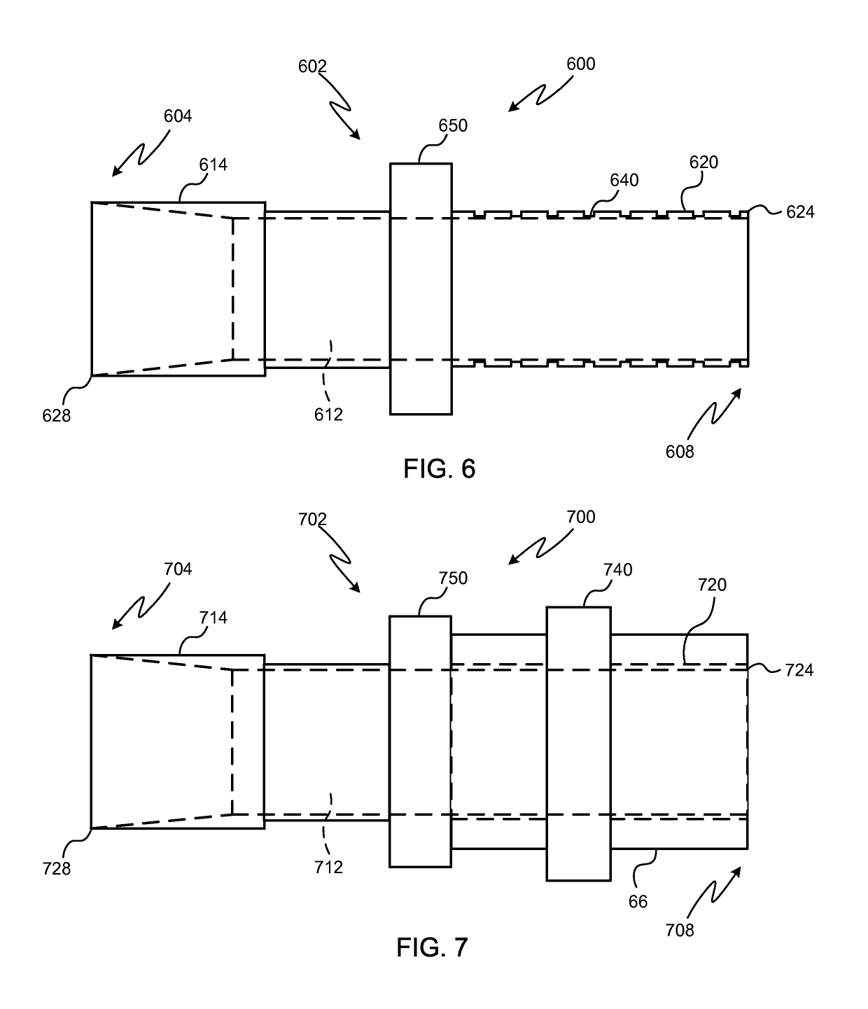

FIG. 6 illustrates one embodiment of a connector 600 that can be incorporated into the blood conduit of the vascular access system 50 of FIG. 1. The connector 600 includes a connector body 602 that has a proximal portion 604, a distal portion 608, and a lumen 612 extending therebetween. The lumen 612 can take any suitable form.

The proximal portion 604 preferably is configured to interface with (e.g., be coupled to) the first conduit 62. The connection between the connector 600 and the conduit 62 can be achieved in any suitable manner. For example, the proximal portion 604 can have an enlarged portion 614 over which the first conduit 62 can be advanced. In other embodiments, the enlarged portion 614 is omitted. Other techniques and structures for connecting the connector 600 and the first conduit 62 may be used herein.

The distal portion 608 is configured to interface with the second conduit 66. In one embodiment, the distal portion 608 includes an outer surface 620 that extends between a distal end 624 and a proximal end 628 of the connector 600. In some instances, the outer surface 620 extends from the distal end 624 to a proximal end of the distal portion 608, adjacent to an enlarged segment 650. The connector 600 includes one or more channels 640 positioned about the outer surface 620. In some instances, a plurality of channels 640 may extend about the outer surface 620. Although channels 640 are illustrated, the outer surface 620 may also include one or more holes, divots, grooves, notches, slots, etc., or a combination thereof. The size, position, and shape of the channels 640 may vary. For example, the channels 640 may extend wholly or partially about the perimeter of the outer surface 620. Further, the channels 640 may extend radially and/or axially about the outer surface 620. The channels 640 may extend about the outer surface 620 in any pattern, such as a helix or the like. In this manner, a proximal portion of the second conduit 66 may be configured to be slid over the channels 640 about the outer surface 620. The channels 640 may facilitate maintaining an attachment between the proximal portion of the second conduit 66 and the connector 600. In some instances, the proximal portion of the second conduit 66 include one or more protrusions or ribs that correspond to the channels 640. Other attachments means described herein may be used in combination with the channels 640 to secure the second conduit 66 to the connector 600.

FIG. 7 illustrates one embodiment of a connector 700 that can be incorporated into the blood conduit of the vascular access system 50 of FIG. 1. The connector 700 includes a connector body 702 that has a proximal portion 704, a distal portion 708, and a lumen 712 extending therebetween. The lumen 712 can take any suitable form.

The proximal portion 704 preferably is configured to interface with (e.g., be coupled to) the first conduit 62. The connection between the connector 700 and the first conduit 62 can be achieved in any suitable manner. For example, the proximal portion 704 can have an enlarged portion 714 over which the first conduit 62 can be advanced. In certain embodiments, the enlarged portion 714 is omitted. Other techniques and structures for connecting the connector 700 and the first conduit 62 may be used herein.

The distal portion 708 is configured to interface with the second conduit 66. In one embodiment, the distal portion 708 includes an outer surface 720 that extends between a distal end 724 and a proximal end 728 of the connector 700. In some instances, the outer surface 720 extends from the distal end 724 to a proximal end of the distal portion 708, adjacent to an enlarged segment 750. The connector 700 includes one or more connection devices 740 positioned about the outer surface 720 so as to secure a proximal portion of the second conduit 66 to the connector 700. The size, position, and shape of the connection devices 740 may vary. In this manner, the proximal portion of the second conduit 66 may be configured to be slid over the outer surface 720, and the connection devices 740 is positioned/positionable about the proximal portion of the second conduit 66 so as to secure it to the outer surface 720. The connection devices 740 may facilitate maintaining an attachment between the proximal portion of the second conduit 66 and the connector 700. In some instances, the connection devices 740 include one or more clamps, ties, compression tubes, mounts, combinations thereof, or the like. In certain embodiments, the connection devices 740 are a separate component from the connector 700. That is, the connection devices 740 may be external (or extrinsic) to the connector 700. In other embodiments, the connection devices 740 are integral with the connector 700. For example, the connection devices 740 may comprise one or more clamps described in U.S. Application Publication No. 2013/0060268. Other attachments means described herein may be used in combination with the connection devices 740 to secure the second conduit 66 to the connector 700.

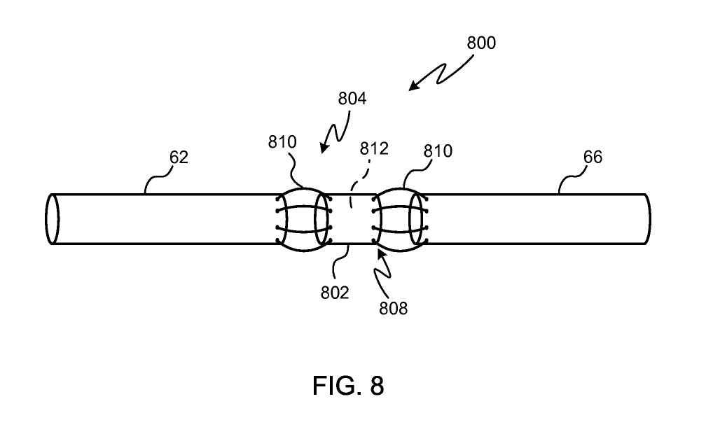

FIG. 8 illustrates an embodiment of a connector 800 that can be incorporated into the blood conduit of the vascular access system 50 of FIG. 1. The connector 800 includes a connector body 802 that has a proximal portion 804, a distal portion 808, and a lumen 812 extending therebetween. The lumen 812 can take any suitable form.

The proximal portion 804 is configured to interface with (e.g., be coupled to) the first conduit 62. The connection between the connector 800 and the first conduit 62 can be achieved in any suitable manner. For example, the proximal portion 804 can be attached to the first conduit 62 by way of one or more sutures 810 or the like. Other techniques and structures for connecting the connector 800 and the first conduit 62 may be used herein. The distal portion 808 is configured to interface with the second conduit 66. The connection between the connector 800 and the second conduit 66 can be achieved in any suitable manner. For example, the distal portion 808 can be attached to the second conduit 66 by way of one or more sutures 810 or the like. Other techniques and structures for connecting the connector 800 and the second conduit 66 may be used herein.

The various apparatuses, systems, and methods associated with the connectors described in FIGS. 2-8 may be interchanged. That is, it is within the scope of the disclosure that certain embodiments of the connectors described herein may include components, features, and/or functions of any of the other connectors disclosed. For example, any of the connectors described herein may comprise, among other things, one or more barbs, protrusions, voids, channels, textured surfaces, clamps (both integral and/or external), sutures, or combinations thereof. Further, the various apparatuses, systems, and methods may be used to secure the first conduit 62, such as a graft, to the connectors described herein. That is, the proximal portions of the connectors may include, among other things, one or more barbs, protrusions, voids, channels, textured surfaces, clamps (both integral and/or external), sutures, or combinations thereof.

The various apparatuses, systems, and methods associated with the connectors described in FIGS. 2-8 may provide several technical advantages over the prior art. For example, the various engagement features of the connectors may facilitate ease of manufacturability of the connector, ease of manufacturability of the graft/connector assembly, greater security/performance of the connection, and/or provide the potential for easier disassembly in case of revision or replacement. Other advantages may become apparent throughout the disclosure.

The foregoing method of connecting an inflow component to any of the connectors herein can be embedded within a broader method of applying such a device to a patient. Such a method can involve accessing a vein in the patient into which an outflow component is to be inserted. For example, a jugular vein could be accessed in such a method. The distal end of the outflow component can be positioned distant from the access site, e.g., at any location between the access site to a chamber of the heart.

The proximal end of the outflow component can thereafter be positioned at any suitable anatomical location, e.g., at the nearest delta-pectoral groove. Such positioning of the proximal end of the outflow component can be achieved in any suitable manner, such as by tunneling subcutaneously the proximal end from adjacent to the venous insertion site to adjacent to the delta-pectoral groove.

The broader method can also include coupling the proximal end of an inflow component with a vascular segment different from the insertion site, which can be a jugular vein. The inflow component can be coupled with a different vascular segment, e.g., a brachial artery by any suitable technique. One technique involves suturing the proximal end to the artery, for example producing an end-to-side anastomosis. Attachment of the proximal end of the inflow component can be performed through a second incision formed through the skin adjacent to the vascular segment to which the inflow component is to be connected. After connecting the inflow component, the distal end of the inflow component can be tunneled, e.g., subcutaneously to an anatomical location suitable for coupling to the outflow component, e.g., to the delta-pectoral groove, where a third incision can be formed.

Once distal and proximal portions of the inflow and outflow components respectively are located at a connection zone (e.g., delta-pectoral groove), connection of these components can be achieved using the connector apparatuses, systems, and methods discussed above. In one convenient technique, a short length of a distal portion of the inflow component is lifted out of the patient through the third incision and a short length of a proximal portion of the outflow component is lifted out of the patient through the third incision. The proximal end of the outflow component is advanced over the distal tubular structure of the connector including, in some instances, the engagement features (or similar, as discussed herein). The distal end of the inflow component is advanced over the proximal tubular structure of the connector. A variety of methods of this type are discussed in general in U.S. Pat. No. 7,762,977, which is hereby incorporated by reference for the purpose of elaborating on these techniques.

The foregoing devices and variants thereof enable the provision of a vascular access system. The inflow component can be attached by any means to an artery by suturing or otherwise arterialized. In other embodiments, the proximal end of the inflow component is attached by an expandable member, which can be self-expanding or balloon expandable. A self-expanding version can include a sinusoidal circumferential member adapted to be enlarged to at least the inner size of the artery. This enlargement enables a proximal portion of the inflow conduit to expand toward the inner wall of the artery, e.g., to be pressed into engagement with an internal segment of an artery. Another technique for arterializing the inflow component involves providing a coupling structure, which can be one or more stent-like structures, such as those described for example in U.S. Application Publication No. 2009/0076587, which is incorporated by reference herein in pertinent part. For example, at least a portion of the inflow component, e.g., including at least a portion of the coupling structure, can be deployed within the vessel and the remainder of the inflow component can extend from the vessel to the connector.

A portion of the outflow component is adapted to be inserted within a vein at an insertion site. The outflow component can have an outside diameter which is less than an inner diameter of the vein and can have at least one opening in an end thereof with at least one of the openings in the catheter section to be disposed distant from the insertion site. For example, an outlet can be in the heart.

In operation, blood flows from the artery through the catheter and is returned to the venous side of the circulatory system through an opening in the outflow component. The system preferably provides laminar blood flow between the artery and the vein. In certain applications, blood flows through the vein uninterrupted around at least an outer portion of the outflow component.

Access to the system can be provided in any suitable way, such as by providing a needle having a first end coupled to a hemodialysis device and having a second end adapted for insertion directly into the inflow component. Blood may thereby be shunted from the vascular access device to a dialysis device and back to the patient's circulatory system.

Although disclosed in the context of certain preferred embodiments and examples, it will be understood by those skilled in the art that the present disclosures extend beyond the specifically disclosed embodiments to other alternative embodiments and/or uses of and obvious modifications and equivalents thereof. In addition, while several variations have been shown and described in detail, other modifications, which are within the scope of these disclosures, will be readily apparent to those of skill in the art. It is also contemplated that various combinations or sub-combinations of the specific features and aspects of the embodiments may be made and still fall within the scope of the disclosures. It should be understood that various features and aspects of the disclosed embodiments can be combined with or substituted for one another in order to form varying modes of the disclosed embodiments. Thus, it is intended that the scope of at least some of the present embodiments herein disclosed should not be limited by the particular disclosed embodiments described above.

We claim:

1. A system for providing continuous flow of blood between two locations in a cardiovascular system of a patient, the system comprising:
a blood conduit having a distal portion and a proximal portion, the distal portion configured to be inserted into a blood vessel at an insertion site and to be advanced therethrough to a location spaced apart from the insertion site; and
a connector comprising a distal portion configured to be engaged with the proximal portion of the blood conduit, whereby continuous flow can be provided through the connector and into the blood conduit, wherein the connector comprises one or more engagement features disposed about at least a portion of an outer surface of the distal portion of the connector, wherein the one or more engagement features comprise one or more textured surfaces disposed about all or part of the outer surface of the distal portion of the connector.

2. The system of claim 1, wherein the one or more textured surfaces comprise a pattern disposed about all or part of the outer surface of the distal portion of the connector.

3. The system of claim 1, wherein the one or more engagement features further comprise one or more: barbs, channels, holes, divots, protrusions, sutures, extrinsic connection devices, integral connection devices, or combinations thereof.

4. A system for providing continuous flow of blood between two locations in a cardiovascular system of a patient, the system comprising:
a blood conduit having a distal portion and a proximal portion, the distal portion configured to be inserted into a blood vessel at an insertion site and to be advanced therethrough to a location spaced apart from the insertion site; and
a connector comprising a distal portion configured to be engaged with the proximal portion of the blood conduit, whereby continuous flow can be provided through the connector and into the blood conduit, wherein the connector comprises one or more engagement features disposed about at least a portion of an outer surface of the distal portion of the connector, wherein the one or more engagement features comprise one or more channels disposed about all or part of the outer surface of the distal portion of the connector.

5. The system of claim 4, wherein the one or more channels extend radially about the outer surface of the distal portion of the connector.

6. The system of claim 4, wherein the one or more channels extend axially about the outer surface of the distal portion of the connector.

7. The system of claim 4, wherein the one or more channels extend about the outer surface of the distal portion of the connector in a pattern.

8. The system of claim 7, wherein the pattern is a helix.

9. The system of claim 4, wherein the one or more engagement features further comprise one or more: barbs, textured surfaces, holes, divots, protrusions, sutures, extrinsic connection devices, integral connection devices, or combinations thereof.

10. A connector for implantation in a cardiovascular system, the connector comprising:
a distal tubular body;
a proximal tubular body;
a flange disposed between the proximal and distal tubular bodies, wherein an outer surface of the distal tubular body is configured to be engaged with a proximal portion of a blood conduit; and
one or more engagement features disposed about the outer surface of the distal tubular body, wherein the one or more engagement features comprise one or more discrete protrusions disposed about all or part of the outer surface of the distal tubular body.

11. The connector of claim 10, wherein the one or more discrete protrusions are disposed between the flange and a distal end of the distal tubular body.

12. The connector of claim 10, wherein the one or more discrete protrusions comprise a plurality of discrete protrusion disposed about the outer surface of the distal portion of the connector.

13. The connector of claim 12, wherein the plurality of discrete protrusions are aligned in a uniform manner about the outer surface of the distal portion of the connector.

14. The connector of claim 12, wherein the plurality of discrete protrusions are disposed intermittently about the outer surface of the distal portion of the connector.

15. The connector of claim 10, wherein the one or more engagement features further comprise one or more: barbs, textured surfaces, channels, holes, divots, sutures, extrinsic connection devices, integral connection devices, or combinations thereof.

16. A connector for fluidly coupling proximal and distal tubular segments of a fluid conduit in a cardiovascular system, the connector comprising:
a connector body having an outer surface defining an outer perimeter and an inner surface defining a lumen;
a proximal portion configured to be engaged with the proximal tubular segment of the fluid conduit;
a distal portion configured to be engaged with the distal tubular segment of the fluid conduit; and
one or more engagement features disposed about the distal portion of the connector body, wherein the one or more engagement features comprise one or more extrinsic connection devices disposed about all or part of the outer surface of the distal portion of the connector and configured to attach the proximal portion of the blood conduit to the connector, whereby continuous flow can be provided through the lumen of the connector between the proximal and distal tubular segments of the fluid conduit.

17. The connector of claim 16, wherein the one or more extrinsic connection devices comprise one or more clamps, ties, compression tubes, mounts, or combinations thereof.

18. The connector of claim 16, wherein the one or more engagement features further comprise one or more: barbs, textured surfaces, channels, holes, divots, protrusions, sutures, integral connection devices, or combinations thereof.

19. A connector for implantation in a cardiovascular system, the connector comprising:
a distal tubular body;
a proximal tubular body; and
a flange disposed between the proximal and distal tubular bodies, wherein an outer surface of the distal tubular body is configured to be engaged with a proximal portion of a blood conduit, wherein the outer surface of the distal tubular body is uniform and includes no engagement features.

20. The connector of claim 19, further comprising one or more extrinsic connection devices disposed about all or part of the outer surface of the distal tubular body of the connector and configured to attach the proximal portion of the blood conduit to the connector.
